(19) United States
(12) Patent Application Publication
(10) Pub. No.: US 2015/0335274 A1
(43) Pub. Date: Nov. 26, 2015

(54) PHYSIOLOGICAL SIGNALS DETECTION DEVICE

(71) Applicant: EMINENT ELECTRONIC TECHNOLOGY CORP. LTD., Hsinchu (TW)

(72) Inventors: Tom Chang, Taipei City (TW); Kao-Pin Wu, New Taipei City (TW); Chih-Jen Fang, Tainan City (TW); Shang-Ming Hung, Zhubei City (TW); Chiao-Yi Huang, Zhubei City (TW); Chan-Peng Lo, Taichung City (TW); Tseng Kuo-Tsai, Taoyuan City (TW)

(73) Assignee: EMINENT ELECTRONIC TECHNOLOGY CORP. LTD.

(21) Appl. No.: 14/708,527

(22) Filed: May 11, 2015

Related U.S. Application Data
(60) Provisional application No. 62/002,932, filed on May 26, 2014.

(30) Foreign Application Priority Data
Apr. 13, 2015 (TW) 104111745

(51) Int. Cl. A61B 5/1455; A61B 5/00; A61B 5/024

(52) U.S. Cl. CPC A61B 5/14551; A61B 5/02433; A61B 5/7203; A61B 5/6826

(57) ABSTRACT

A physiological signals detection device has a light source connecting to a control unit, a light detector and a processing unit. The light detector has a pixel sensor array including multiple light sensing elements. The light source emits light through a lens to the human body to generate reflected light. The light detector receives the reflected light to generate a sensing signal. Since the light sensing elements respectively receive different reflected light from different directions, the light sensing elements receiving reflected light from the noise are easily selected and eliminated from calculating the physiology value. Therefore, the calculated physiology value is more accurate.

### CROSS-REFERENCE TO RELATED APPLICATIONS

This application claims the benefit of United States provisional application filed on May 26, 2014 and having application Ser. No. 62/002,932, the entire contents of which are hereby incorporated herein by reference

This application is based upon and claims priority under 35 U.S.C. 119 from Taiwan Patent Application No. 104111745 filed on Apr. 13, 2015, which is hereby specifically incorporated herein by this reference thereto.

### BACKGROUND OF THE INVENTION

1. Field of the Invention

The present invention relates to a physiological signals detection device, especially to a physiological signals detection device using light detectors to detect human bodies' physiological signals.

2. Description of the Prior Arts

With the progress of the technology, more and more functions can be achieved by the light detection. One of the functions is to detect human bodies' physiological signals. Regarding heartbeat rate detection as an example, the heartbeat results in the flow of the blood to further result in the cyclical variation of the blood pressure. The pressure variation of the blood vessel changes the diameter of the blood vessel so that the cyclical variation of the blood pressure causes the diameter of the blood vessel to change continuously. Therefore, using the light detection result of the variation of the diameter of the blood vessel records the light variation of the reflected light from the light source emitting to the blood vessel to further calculate the heartbeat value. Other physiological values such as blood oxygen saturation index, blood pressure and so on can also be further calculated through obtaining the blood vessel signals or the blood signals via light detection.

The conventional physiological signals detection device includes at least one light source and a light detector. The light source provides light beam emitted through the skin to the blood vessel and then results in a reflected light. The reflected light passes through the skin and is received by the light detector. The light detector converts the reflected light into a sensing signal. Then the processing unit calculates the desired physiological signal value based on the sensing signal.

However, with referenced to FIG. 5, the light beam from the light source is also emitted to other objects on the path to the blood vessel, such as epidermal tissue. The light beam emitted to those other objects also results in reflected lights to be received by the light detector 60 together. Since the single light detector 60 receives the reflected lights from all directions, the reflected light resulting from the blood vessel and the reflected light resulting from those other objects are all received by the same light detector 60. The light detector 60 can only converts the sum of the received reflected light into the sensing signal. Therefore, the sensing signal not only includes the information of the desired blood continuously variation value or the desired blood signal, but also includes other noises. Thus, the physiological signal value calculated by the processing unit based on the sensing signal is also not accurate.

To overcome the shortcomings, the present invention provides a physiological signals detection device to mitigate or obviate the aforementioned problems.

### SUMMARY OF THE INVENTION

The main objective of the present invention is to provide a physiological signals detection device that excludes the noise from calculation to improve the accuracy of the physiological values.

The physiological signals detection device comprising:

a first light source providing a first light beam;

a light detector having
a pixel sensor array having multiple first light sensing elements and detecting a reflected light resulted from the first light beam emitted from the first light source to a user's body to generate a first sensing signal;

a lens covering on the light detector, and the pixel sensor array receiving the reflected light of the first light beam through the lens;

a processing unit connecting to the light detector and calculating the user's heartbeat value based on the first sensing signal; and

a control unit connecting to the first light source, the light detector and the processing unit, switching the first light source on and off, controlling the light detector to sense and controlling the processing unit to process signals.

The physiological signals detection device has following advantages. With the pixel sensor array, different light from different directions are received by different light sensing elements so that the control unit can determine whether the received signals are desired signals or a noises based on the intensity, frequency and so on of the received signals. Then the received signals from the light sensing elements that are determined as receiving noises are excluded. Only the received signals from the light sensing elements that are determined as receiving the desired signals are used to calculate the physiological value. Therefore, the noises are excluded from influencing the physiological value to enhance the accuracy of the physiological value.

Other objectives, advantages and novel features of the invention will become more apparent from the following detailed description when taken in conjunction with the accompanying drawings.

### BRIEF DESCRIPTION OF THE DRAWINGS

FIG. 1 is an operational view of a physiological signals detection device in accordance with the present invention, shown a finger being placed on;

FIG. 2 is a block diagram of a circuit of the physiological signals detection device in FIG. 1;

FIG. 3 is an illustrated view of a light detector of the physiological signals detection device in FIG. 1;

FIG. 4 is an illustrated view of a light path for receiving a reflected light by the physiological signals detection device in FIG. 1; and

FIG. 5 is an illustrated view of a light path for receiving a reflected light by a conventional physiological signals detection device in accordance with the prior art.

### DETAILED DESCRIPTION OF THE PREFERRED EMBODIMENTS

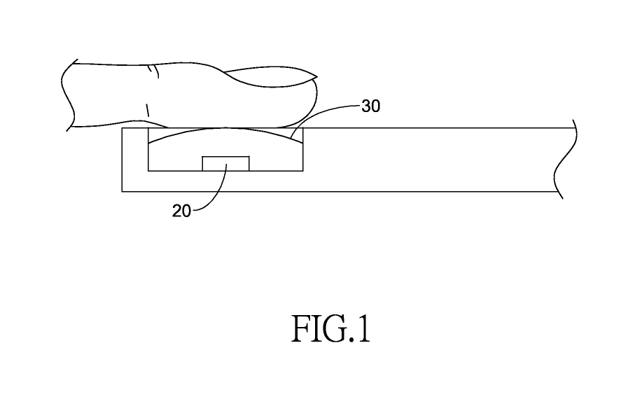

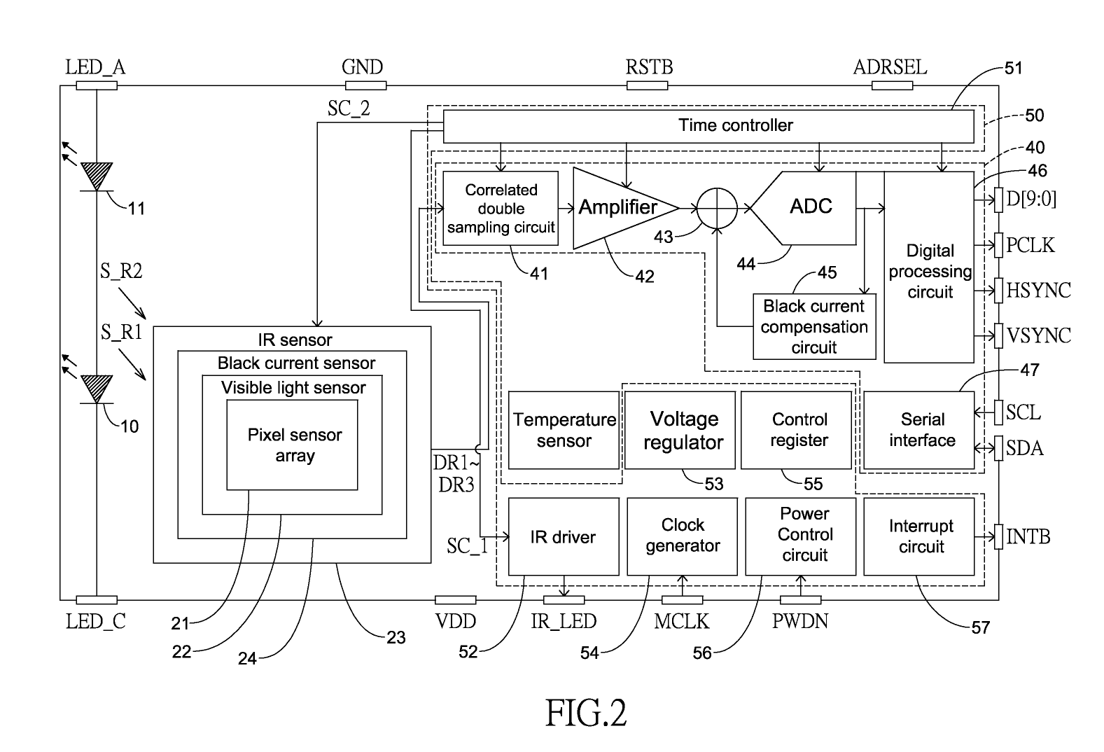

With reference to FIGS. 1 and 2, a physiological signals detection device in accordance with the present invention comprises a first light source 10, a light detector 20, a lens 30, a processing unit 40 and a control unit 50.

The first light source 10 provides a first light beam. The first light beam may be infrared (IR) light. The physiological signals detection device further comprises a second light source 11 to provide a second light beam. The wavelength of the second light beam is different with the wavelength of the first light beam. The second light beam may be a green light. When the physiological signals detection device as described is applied to detect the heartbeat value or the blood pressure, the physiological signals detection device may only use a single light source such as the first light source 10 or the second light source 11. When the physiological signals detection device as described is applied to detect the blood oxygen saturation index, the blood oxygen saturation index as described needs to use both the first and second light sources 10, 11 with different wavelength.

The light detector 20 has a pixel sensor array 21 with multiple light sensing elements 211. Each light sensing element 211 detects a reflected light resulting from the first light beam emitted to the user's body and generates a corresponding sensing signal.

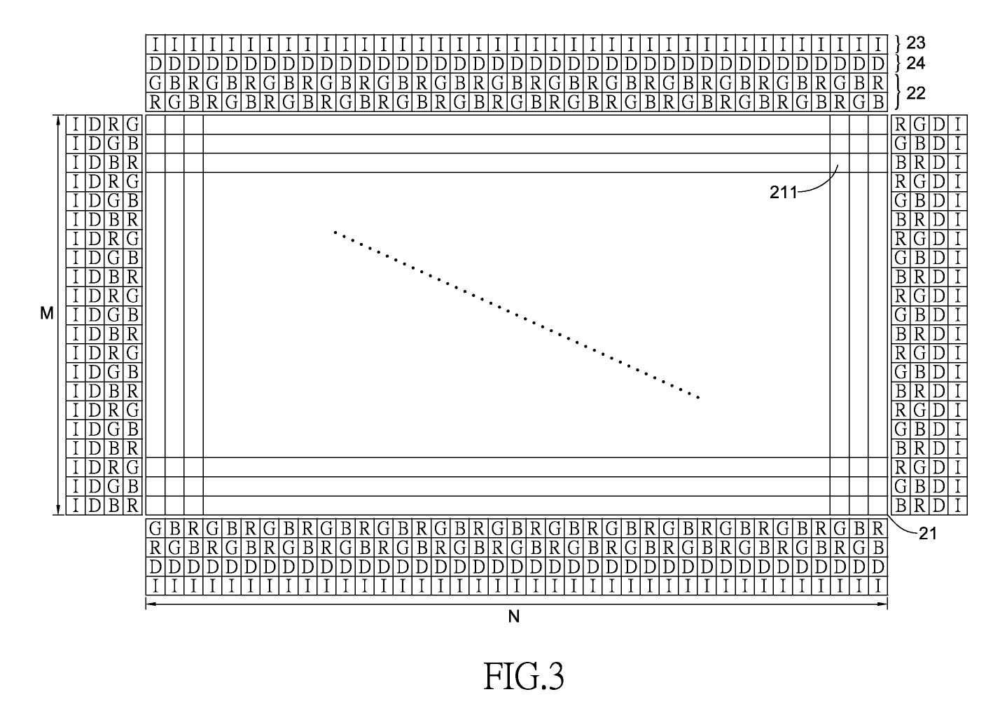

With reference to FIG. 3, in one embodiment, the pixel sensor array 21 may comprises M×N light sensing elements 211. M and N are positive integer. The pixel sensor array 21 may be an active pixel sensor array. The light sensing elements 211 may comprises multiple first light sensing elements and multiple second sensing elements. The first light sensing elements are used to detect the reflected light of the first light beam. The second light sensing elements are used to detect the reflected light of the second light beam. In one embodiment, the light detector 20 further comprises a visible light sensor 22, an IR sensor 23 and a black current sensor 24. The visible light sensor 22 is mounted adjacent to the pixel sensor array 21 to perform ambient light sensing and/or color sensing. The black current sensor 24 is mounted adjacent to the pixel sensor array 21 to generate a reference signal for dark/black current compensation. The IR sensor 23 is mounted adjacent to the pixel sensor array 21 to perform proximity sensing, object position sensing, and/or gesture sensing. In one embodiment, the black current sensor 24 is mounted around the visible light sensor 22, the IR sensor 23 is mounted around the black current sensor 24. However, the arrangement is illustrated only and does not limit the present invention. For example, when the IR sensor 23 is mounted between the visible light sensor 22 and the pixel sensor array 21, the functions of the pixel sensor array 21, the visible light sensor 22, the IR sensor 23 and the black current sensor 24 are not influenced.

The lens 30 covers the light detector 20. The light detector 20 senses light through the lens 30.

The processing unit 40 is connected to the light detector 20 to process the sensing signal of the light detector 20 and to further calculate the desired physiological signal such as the heartbeat value, the blood oxygen saturation index, the blood pressure and so on. In one embodiment, the processing unit 40 comprises, but is not limited to, a correlated double sampling circuit 41, an amplifier 42, an adder 43, an analog-to-digital converter 44, a black current compensation circuit 45, a digital processing circuit 46 and a serial interface 47 (serial I/F such as two wire inter-integrated circuit). The signals out from the pixel sensor array 21 are processed by the correlated double sampling configuration with programmable gain settings consisted of the correlated double sampling circuit 41 and the amplifier 42. The adder 43 adds the output of the amplifier 42 and the output of the black current compensation circuit 45 into an analog signal as an output of the adder 43. Then the analog-to-digital converter 44 converts the analog signal to a digital signal as an output of the analog-to-digital converter 44. The output of the black current compensation circuit 45 is generated based on the digital signal. The digital processing circuit 238 performs further operations upon the digital signal (e.g. the threshold comparison, the hysteresis detection and other detection algorithms), and transmits resulting data through the multiple contacts D [9:0], PCLK, HSYNC and VSYNC. The serial interface 47 is used for synchronous serial communication between the chips, and is coupled to a contact SCL corresponding to a serial clock line and a contact SDA corresponding to a serial data line. Since a person skilled in the art should understand the operations of each circuit element included in the processing unit 40, no detailed discussion is further disclosed.

The control unit 50 is connected to the first light source 10, the second light source 11, the light detector 20 and the processing unit 40, The control unit 50 switches the first light source 10 and the second light source 11 on and off, controls the light detector 20 to sense, and controls the signal processing of the processing unit 40. In one embodiment, the control unit 50 comprises, but is not limited to, a timing controller 51, an IR LED driver 52, a voltage regulator 53, a clock generator 54, a control register 55, a power control circuit 56 and an interrupt circuit 57. The timing controller 51 generates the control signal S_C1 to control the IR LED driver 52, and generates the control signal S_C2 to control the pixel sensor array 21. The IR LED driver 52 activates and deactivates the first light source 10 according to the control signal S_C1. The clock generator 54 receives an external clock such as a master clock from a contact MCLK. The power control circuit 56 receives a power control signal from a contact PWDN to control a power operation mode. The interrupt circuit 57 receives an interrupt signal from a contact INTB. Since a person skilled in the art should understand the operations of each circuit element included in the control unit 50, no detailed discussion is further disclosed.

In one embodiment, when the control unit 50 switches off the pixel sensor array 21, the control unit 50 may switch on the visible light sensor 22 to perform ambient light sensing and/or color sensing. Similarly, when the control unit 50 switches off the pixel sensor array 21, the control unit 50 may switch on the IR sensor 23 to perform proximity sensing, object position sensing, and/or gesture sensing. Therefore, when no physiological signals detection is needed, the pixel sensor array 21 may be switched off to save power but the visible light sensor 22 and the IR sensor 23 are still functioned to proceed detection with lower power consumption. Therefore, reducing power consumption is achieved.

With reference to FIGS. 1 and 2, when the physiological signals detection device as described is used to detect heartbeat value, the user puts the corresponding part such as finger on the top of the lens 30. The first light source 10 emits the first light to the finger to generate the reflected light, and then the reflected light pass through the lens 30 to be received by the light detector 20.

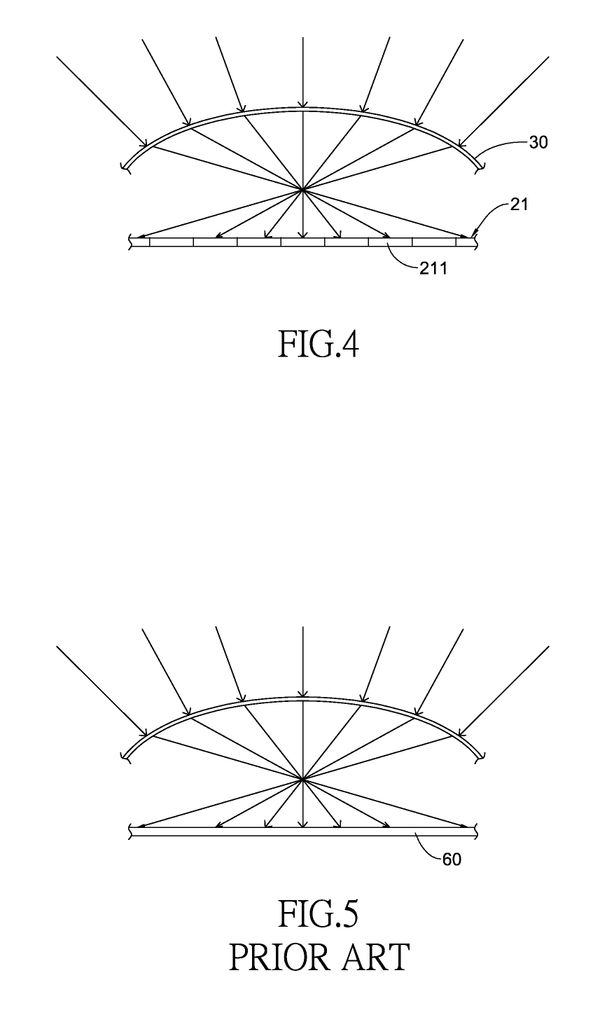

With reference to FIGS. 2 and 4, since the pixel sensor array 21 of the light detector 20 has multiple light sensing elements 211, each light sensing element 211 receives a reflected light from one direction. Different light sensing elements 211 receive the reflected light from different direction. Each light sensing element 211 transmits the sensing signal to the processing unit 40. The control unit 50 determines each received sensing signal is desired signal or noise based on the character of the reflected light, and controls the signal operation of the processing unit 40. As to the light sensing elements 211 receive the desired signal, the processing unit 40 calculates the corresponding physiological value based on their sensing signals. As to the light sensing elements 211 receive the noises, the processing unit 40 ignores their sensing signals.

Therefore, with the pixel sensor array 21 having multiple light sensing elements 211, the light sensing elements 211 receiving the desired signals are distinguished from the light sensing elements 211 receiving the noises. Thus, the noises are easily excluded from the further calculation to allow the output physiological signals to be more precise.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A physiological signals detection device comprising:
a first light source providing a first light beam;
a light detector having
a pixel sensor array having multiple first light sensing elements and detecting a reflected light resulted from the first light beam emitted from the first light source to a user's body to generate a first sensing signal;
a lens covering on the light detector, and the pixel sensor array receiving the reflected light of the first light beam through the lens;
a processing unit connecting to the light detector and calculating the user's heartbeat value based on the first sensing signal; and
a control unit connecting to the first light source, the light detector and the processing unit, switching the first light source on and off, controlling the light detector to sense and controlling the processing unit to process signals.

2. The physiological signals detection device as claimed in claim 1 further comprising a second light source, wherein
the control unit connects to and switches the second light source on and off;
the second light source provides a second light beam having a wavelength different from a wavelength of the first light beam;
the pixel sensor array further has multiple second light sensing elements and detects a reflected light resulted from the second light beam emitted from the second light source to a user's body to generate a second sensing signal; and
the processing unit calculates the blood oxygen saturation index based on the first and second sensing signals.

3. The physiological signals detection device as claimed in claim 1, wherein the first light beam is infrared light.

4. The physiological signals detection device as claimed in claim 2, wherein the first light beam is an infrared light and the second light beam is a green light.

5. The physiological signals detection device as claimed in claim 1, wherein
the light detector further has a visible light sensor receiving a visible light, generating a third sensing signal and mounted adjacent to the pixel sensor array;
the control unit connects to and controls the visible light sensor to sense; and
the processing unit calculates an illumination a color contrast of an ambient light based on the third sensing signal.

6. The physiological signals detection device as claimed in claim 2, wherein
the light detector further has a visible light sensor receiving a visible light, generating a third sensing signal and mounted adjacent to the pixel sensor array;
the control unit connects to and controls the visible light sensor to sense; and
the processing unit calculates an illumination a color contrast of an ambient light based on the third sensing signal.

7. The physiological signals detection device as claimed in claim 5, wherein when the pixel sensor array is switched off, the control unit actuates the visible light sensing unit.

8. The physiological signals detection device as claimed in claim 6, wherein when the pixel sensor array is switched off, the control unit actuates the visible light sensing unit.

9. The physiological signals detection device as claimed in claim 1, wherein
the light detector further has an infrared sensor receiving a reflected light from an infrared light, generating a fourth sensing signal and mounted adjacent to the pixel sensor array;
the control unit connects to and controls the infrared sensor to sense; and
the processing unit calculates whether an object approaches, to determine a position of the object or to determine a gesture based on the fourth signal.

10. The physiological signals detection device as claimed in claim 2, wherein
the light detector further has an infrared sensor receiving a reflected light from an infrared light, generating a fourth sensing signal and mounted adjacent to the pixel sensor array;
the control unit connects to and controls the infrared sensor to sense; and
the processing unit calculates whether an object approaches, to determine a position of the object or to determine a gesture based on the fourth signal.

11. The physiological signals detection device as claimed in claim 5, wherein
the light detector further has an infrared sensor receiving a reflected light from an infrared light, generating a fourth sensing signal and mounted adjacent to the pixel sensor array;
the control unit connects to and controls the infrared sensor to sense; and
the processing unit calculates whether an object approaches, to determine a position of the object or to determine a gesture based on the fourth signal.

12. The physiological signals detection device as claimed in claim 9, wherein when the pixel sensor array is switched off, the control unit actuates the infrared sensor.

13. The physiological signals detection device as claimed in claim 10, wherein when the pixel sensor array is switched off, the control unit actuates the infrared sensor.

14. The physiological signals detection device as claimed in claim 11, wherein when the pixel sensor array is switched off, the control unit actuates the infrared sensor.

15. The physiological signals detection device as claimed in claim 1, wherein the light detector further has an black current sensor mounted adjacent to the pixel sensor array and connecting to the control unit and the processing unit to generate a reference signal for black current compensation.

16. The physiological signals detection device as claimed in claim 2, wherein the light detector further has an black current sensor mounted adjacent to the pixel sensor array and connecting to the control unit and the processing unit to generate a reference signal for black current compensation.

17. The physiological signals detection device as claimed in claim 5, wherein the light detector further has an black current sensor mounted adjacent to the pixel sensor array and connecting to the control unit and the processing unit to generate a reference signal for black current compensation.

18. The physiological signals detection device as claimed in claim 9, wherein the light detector further has an black current sensor mounted adjacent to the pixel sensor array and connecting to the control unit and the processing unit to generate a reference signal for black current compensation.

19. The physiological signals detection device as claimed in claim 11, wherein the light detector further has an black current sensor mounted adjacent to the pixel sensor array and connecting to the control unit and the processing unit to generate a reference signal for black current compensation.
